(19) United States
(12) Patent Application Publication
(10) Pub. No.: US 2016/0206771 A1
(43) Pub. Date: Jul. 21, 2016

(54) Anti-microbial wound dressing and a method of producing the same

(71) Applicants: Ilpo Pyykkö, Vantaa (FI); Pertti Nousiainen, Vantaa (FI)

(72) Inventors: Ilpo Pyykkö, Vantaa (FI); Pertti Nousiainen, Vantaa (FI)

(73) Assignee: SILVERGREEN OY LTD, Vantaa (FI)

(21) Appl. No.: 13/979,658

(22) PCT Filed: Jan. 19, 2012
(86) PCT No.: PCT/FI2012/050048

§ 371 Date: Jul. 30, 2014

Related U.S. Application Data
(60) Provisional application No. 61/433,987, filed on Jan. 19, 2011.

(30) Foreign Application Priority Data
Jan. 19, 2011 (FI) 20115052

(51) Int. Cl. A61L 15/18; D04H 1/492; A61L 15/26; A61F 13/00; A61L 15/44

(52) U.S. Cl. CPC A61L 15/18; A61F 13/00008; A61F 13/00063; A61F 13/00042; A61L 15/44; A61L 15/26; D04H 1/492; A61F 2013/00936; A61L 2420/02; A61L 2300/606; A61L 2300/404; A61L 2300/104; A61L 2300/102

(57) ABSTRACT

Anti-microbial wound dressing and method of producing the same. The wound dressing comprises a non-woven fabric produced by hydroentanglement of a blend of fibres, said blend being formed by first fibres which are coated with elemental silver or contain silver and second fibres which are essentially free from silver. The wound dressing will retain its antimicrobial properties over extended periods of time, and it is suitable for sustained release of silver.

The present invention concerns an anti-microbial wound dressing according to the preamble of claim 1.

A wound dressing of the present kind typically comprises a non-woven fabric produced by hydroentanglement of a blend of fibres.

The present invention also concerns a method of producing an anti-microbial wound dressing according to the preamble of claim 25.

Wound dressings are primarily formed in four different ways:
a. Traditional wound dressings are made of absorbent textile materials, such as cotton and viscose, and they are produced by knitting or by mechanical or mechano-chemical non-woven methods;
b. Wound dressings of great moisture absorbing capacity which include a fibrous layer, preferably gel-forming fibres, incorporated into the fibrous matrix, are formed by cotton or viscose fibres, as disclosed in EP Patent No. 1 314 410;
c. Wound dressings based on foamed polymers which can be combined with the use of antimicrobial additives, such as silver ions (Ag+) or other antimicrobial substances; and
d. Gelled wound dressing into which antimicrobial additives, such as metal salts, chitosan or organic antimicrobial compounds can be incorporated.

The above alternatives can also be combined. Further, each layer can be made thicker and it can be rendered more absorptive. For making a wound dressing suitable for the actual need, i.e. for each individual wound, usually a combination of the above solutions has to be employed for attaining sufficient properties of liquid wicking, liquid absorption and retention, liquid evaporation, antimicrobial activity and mechanical strength. Thus, typically, based on the above alternatives liquid which is wicked into the wound dressing can be evaporated (alternative a) or it can be absorbed into an absorptive gel (alternatives b to d).

A problem relating to alternative a) is that the material is mostly somewhat rough or “furry”. This gives rise to a risk that the wound dressing may stick to the healing wound tissue during scar-forming. Similarly, in alternative b) reinforcing fibres are used for improving the mechanical strength of the wound dressing—such fibres also tend to stick to the scarring tissue. Further, in alternative b) there is a need for a large amount of fibres capable of absorbing moisture for preventing maceration of the wound. In all four alternatives a) to d), any antimicrobial components has to be added separately, for example by depositing a silver compound on the fabric.

To reduce the tendency of the wound dressing sticking to the healing would, various films or polyurethane foams typically are used. The absorptive properties are achieved by incorporating gels which have poor mechanical properties which then calls for the use of additional fibrous structures which are capable of ensuring proper strength.

For the above-mentioned technical reasons a material has been sought which can be used in various situations where there is a need for wound dressings and which meets requirements of absorption, liquid retention, liquid evaporation, antimicrobial activity, smoothness and mechanical strength properties.

WO 0224240 discloses a method of producing wound dressing which contains first fibres capable of bonding with silver cations and second fibres which are substantially free from silver. The first fibres are insoluble in water and comprise, for example, CMC fibres. by needle-punching, non-woven fabrics from a mixture of fibres which contain silver and fibres which do not contain silver. The silver ions are introduced into the CMC fibres by ion-exchange. The fibrous CMC is in the form of staple fibres or continuous filament yarn. It can be contained within a textile article such as a nonwoven fabric. The CMC fibre is not particularly strong which limits the manufacturing methods available for the fabric. Further, the fibres are apt to swell and dissolve in aqueous phase, which reduces the applicability of the nonwoven fabric as a wound dressing.

It is an aim of the invention to eliminate at least a part of the problems of the prior art and to provide novel kinds of wound dressings and methods of producing the same.

The present invention is based on the idea of producing a non-woven fabric from a blend of fibres, which comprises at least two kinds of fibres, viz. first fibres, which are coated with elemental silver or which contain silver particles, and second fibres which are essentially free from silver. The silver-free fibres can be of a type which typically confers properties of mechanical strength to the fabric. They can also be of a type which typically confers properties of absorbance to the fabric. Naturally, mixtures of various fibres can equally be used as second fibres.

As an alternative or in addition to silver, the first fibres may contain copper.

The blend of first and second fibres can be deposited on a substrate, such as a belt, a wire mesh or sleeve, and be subjected to mechanical bonding for providing a fabric. In particular, the blend is subjected to hydroentanglement for forming a non-woven fabric which typically exhibits a combination of extended or even permanent antimicrobial properties and good mechanical strength.

More specifically, the present wound dressing is mainly characterized by what is stated in the characterizing part of claims 1 and 23.

The method of producing wound dressings is characterized by what is stated in the characterizing part of claim 25.

By means of the invention, considerable advantages are obtained. Thus, it has been found that a product produced as explained above can be used as such without additional modifications, or possibly in combination with various absorptive materials, in a large number of wound-care situations.

The hydroentanglement technique allows for the production of a non-woven material which contains practically no or low amounts of staple fibre ends, in particular no or low amounts of staple fibre ends, formed from the first fibres, sticking out from the fabric surface; this will reduce or even eliminate the risk of the wound dressing sticking to the healing wound. This technique has not been used in the art for silver-containing fibres. For example the CMC fibres of WO 0224240 are not strong enough for enabling the use of hydroentanglement because that would lead to dissolution of the silver from the fibres into the aqueous phase. The use of hydroentanglement for producing the present wound dressing is highly advantageous since a fabric is produced, which in many cases is even flexible, and which does not stick to the wound. The silver placed inside a thermoplastic polymer will endure processing and it will not be leached out of the fibres afterwards. Rather, the silver will work actively in the form of metallic silver or silver chloride. By varying the proportion of silver-coated to silver-free fibres, desired functional and mechanical properties are readily obtained. In practice, the wound dressing will retain its antimicrobial properties over extended periods of time, and it is suitable for sustained release of silver.

The materials used in the wound dressing do not necessarily need to be sterilized after hydro-entanglement and drying. A wound dressing of the present kind can be washed even with tap water or river water and can be reused after drying which allows for its use in field conditions.

As the below test results indicate, the present wound dressings meet the requirements of, for example, EN ISO 11737-1: 2006. Thus, using the specific materials of the example, a microbial count of only 10 microns per cm³ was reached whereas the standard allows for a maximum microbial count of <200 microns per cm³.

Next the invention will be examined more closely with the aid of a detailed description.

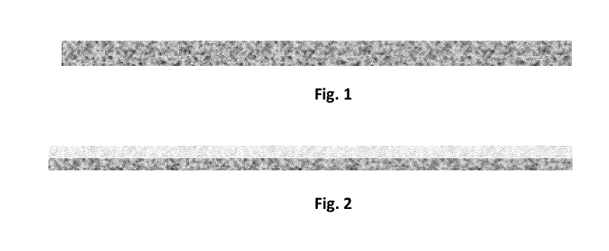

FIG. 1 shows in cross-section a wound dressing according to a first embodiment of the invention, in which silver-coated fibres are evenly distributed throughout the wound dressing; and

FIG. 2 shows in cross-section a wound dressing according to a second embodiment of the invention, in which silver-coated fibres are present primarily in the lower surface (the surface abutting the healing wound).

For the purpose of the present invention, the term “hydroentanglement” stands for a mechanical bonding technique wherein water jets are used to strike a web (a layer of the instant fibre blends) so that the fibres knot about one another, i.e. mechanically bond to each other. Generally, in the art, the term “hydroentanglement” is used synonymously with “spunlacing”.

As discussed above, the novel kind of wound dressings in the form of non-woven fabrics produced by hydroentanglement, comprise first fibres selected from matrix fibres or structural fibres, forming the core of the fibre, which are coated with a layer of elemental silver or which contain silver, for example silver particles, and second fibres which are free from silver.

Within the scope of the present invention, the term “fibres” is to be construed to cover various fibrous and filamentous materials present as individual fibres or fibrils as well as bundles of fibres or fibrils. Said fibres can be present in the shape of yarns (a long continuous length of interlocked fibres) or threads or wires.

The term “silver” is used to designate the metal component of the first fibres. For the sake of order it should be pointed out that other metals can be used also, in particular copper presents an interesting embodiment. Also combinations of the metals can be used, such as silver and copper. Thus, although the following description specifically identifies silver as the metal component, the description is equally applicable to copper.

The fibres (present in any of the forms discussed above) can have a core diameter generally in the range of about 0.01 to 10 μm, preferably about 0.1 to 5 μm, covered with a layer of silver having a thickness of about 1 to 1000 nm, in particular about 5 to 100 nm. The fibres with the previously mentioned dimensions can also contain metallic silver and/or silver salts up to the content mentioned for elementary coating with metallic silver.

The silver-coated fibres typically have a linear density of about 1 to 5 dtex.

The silver coating may enclose the whole perimeter of the fibres or only parts thereof. Generally it is preferred to incorporate into the wound dressing first fibres, at least a part of which have a surface which is fully covered with the silver coating.

The length of the individual fibres can vary, but generally the average length is about 0.01 to 150 mm, in particular about 3 to 100 mm, in particular about 10 to 25 mm.

The core material of the first fibres is formed by a “thermoplastic material” which term is intended to cover polymers and blend of thermoplastic polymers. Examples of suitable polymers include polyolefin homo- and copolymers, such as polyethylene (LDPE, MDPE and HDPE), polypropylene, polybutylene, polyesters (homo- and copolymers), such as polyethylene terephthalate (PET), polybutylene terephthalate (PBT), polytrimethylene terephthalate (PTT) and polyethylene nahpthalate (PEN), as well as biosorbable materials such as polyglycolide (PGA), polylactide (PLA) and polycaprolactone (PCL), polyethylene adipate (PEA), polyamides, such as aliphatic polyamides (PA 6, PA 12 and PA 66), aromatic polyamides, and polyphthalamides, polyacrylics, such as acrylic elastomers and polymethyl methacrylate (PMMA).

According to a first embodiment of the invention, the fibres—potentially in the form of threads, yarns, nonwovens or wires—comprise only one thermoplastic material.

According to a second embodiment of the invention, the fibres—potentially in the form of threads, yarns or wires—comprise blends of two or more of thermoplastic polymers, in particular of thermoplastic polymers mentioned above. The polymers are present in such blends at ratios of about 1:99 to 99:1, preferably about 10:90 to 90:10, in particular 20:80 to 80:20 (based on volume). Blends of polyesters, for example PET, and polyamides (in particular aliphatic, PA 6 or PA 66, or aromatic polyamides) are particularly interesting. The last-mentioned blends contain polyesters and polyamides at ratios of 30:70 to 70:30 (by volume). Blends of polyethylene (HDPE) and polyamides, preferably at the same ratios, are other examples of interesting thermoplastic blends.

The core of the first fibres, formed by the thermoplastic material, is coated with elemental silver. The silver coated fibres comprise about 0.1 to 75%, preferably about 0.5 to 50%, in particular about 0.7 to 35% by weight of elemental silver or silver particles.

The coating of the fibres usable in this application can take place by depositing elemental silver in liquid phase or in gas phase on a substrate formed by the fibrous core material.

Fibres which contain silver deposited on them can also be prepared by manufacturing a silver-containing master-batch formed by silver particles, optionally supported e.g. on ceramic carriers, together with thermo-moldable (thermoplastic) polymer(s) which form silver containing pellets, and by melt-spinning of the thermo-moldable polymers into fibres using said pellets.

During coating, the fibrous core material is typically present in the form of individual fibres or in the form of a thread, yarn or wire.

In liquid phase deposition, silver is precipitated from a liquid medium on fibres optionally in the form of thread, yarn or wire immersed in that liquid with redox properties. The liquid medium may contain silver in the form of finely divided silver particles, as silver ions or by using a suitable reagent, such as Tollens' reagent.

For gas-phase deposition of silver on fibres various techniques are available. Thus, preparation of thermoplastic Ag-loaded fibres can be achieved by RF-plasma and vacuum-UV (V-UV) surface activation followed by chemical reduction of silver salts. It is possible to activate the surface of the fibres before deposition by corona treatment.

According to one embodiment, silver is precipitated on thermoplastic fibres by a wet processing process. Such a process may comprise wet processing of fine polyamide filaments in a yarn dyeing apparatus with a silver solution—or in similar equipment for treatment of yarns or threads—using combined or separate solution treatments with chemical reduction properties. The porous surface structure of the polyamide is remarkably well filled with silver, which increases the amount and durability of the silver layer close to the fibre sheath section, the thickness being up to several micrometers (typically about 0.5 to 10 um). The manufacture of staple fibres can be carried out by cutting the previous filaments or by using a fiber mass in loose form in a mass dyeing machine.

In one embodiment, the first fibres are used in the form of filaments.

In addition to the silver-coated fibres, forming the “first” fibres of the fabric, the present fabric also comprises second fibres which confer the fabric properties of mechanical strength, absorbance and combinations thereof. The second fibres, which typically are silver-free, can be selected from the group of synthetic and natural, hydrophilic fibres, water absorbing films and hydrogels, hydrophobic fibres and combinations thereof

According to one embodiment, the second fibres are selected from the group of hydrophilic and hydrophobic synthetic fibres, in particular hydrophobic thermoplastic fibres, preferably reinforcing fibres capable of conferring properties of mechanical strength and cohesion to the wound dressing.

In such an embodiment, the reinforcing fibres are typically selected from the group of polyolefins, polyacrylics, polyesters and polyamides and mixtures thereof. Examples of suitable polymers include polyolefin homo- and copolymers, such as polyethylene (LDPE, MDPE and HDPE), polypropylene, polybutylene, polyesters (homo- and copolymers), such as polyethylene terephthalate (PET), polybutylene terephthalate (PBT), polytrimethylene terephthalate (PTT) and polyethylene nahpthalate (PEN), as well as biosorbable materials such as polyglycolide (PGA), polylactide (PLA) and polycaprolactone (PCL), polyethylene adipate (PEA), polyamides, such as aliphatic polyamides (PA 6, PA 12 and PA 66), aromatic polyamides, and polyphthalamides, polyacrylics, such as acrylic elastomers and polymethyl methacrylate (PMMA).

According to another embodiment, the second, silver-free fibres are selected from the group of natural and synthetic hydrophilic fibres capable of absorbing liquids.

In such an embodiment, the absorptive fibres are selected from cellulose or viscose fibres, optionally chemically modified or derivatized to modify and optionally increase their absorptive capacity.

The linear density of the second fibres is typically about 0.5 to 5 dtex.

As was discussed above, it is possible to produce a non-woven fabric from a mixture of silver-free fibres, a first portion of which are fibres capable of conferring properties of mechanical strength to the fabric, and a second portion of which are fibres capable of conferring properties of liquid absorbance.

In the fabric according to the present invention, the first fibres form about 1 to 60%, preferably about 5 to 50% of the surface weight of the non-woven fabric.

In one embodiment, second fibres which comprise absorptive fibres form about 10 to 99%, preferably about 20 to 95% of the surface weight of the non-woven fabric. In another embodiment, second fibers which comprise reinforcing fibres form 1 to 60%, preferably 5 to 50% of the surface weight of the non-woven fabric.

According to a preferred embodiment, the surface weight of the non-woven fabric is 30 to 200 g/m², preferably about 40 to about 80 g/m².

The elemental silver is slowly released in ionic form from the fibre surface of the first fibres to the surrounding at a release rate of 1 to 100 ppm/day, preferably about 10 to 70 ppm/day. The release rate is calculated from the total content of silver on the fibres available for release.

The present invention provides a method of producing an anti-microbial wound dressing comprising a non-woven fabric, which method includes the steps of
providing first fibres coated with elemental silver or containing silver throughout the fiber thickness;
providing second fibres which are essentially free from silver;
forming a blend of said first and second fibres; and
producing a non-woven fabric from said blend by hydroentanglement.

Preferably, the first and the second fibres are formed into a fibrous blend on a web for example supported by a wire mesh; and the web is subjected to hydroentanglement process with large number injector needles of high-pressure water jets. At a dimetre of 100 to 170 μm the highest number of needles is up to 1700 needles per meter of injector of high-pressure water jets. The formed web can be run by controlled velocity, using repeated runs and one sided or turned two sided for achieving the desired form of the fabric.

In a particular embodiment, the fibrous blend comprises
1 to 60 parts by weight of first fibres and
40 to 99 parts by weight of second fibres.

The hydroentanglement may further comprise the steps of prewetting a fibrous web; and water-needling said prewetted web using water jets having a pressure of up to 600 bar. Pressures of about 10 to 500 bar can, in principle, be used. After the hydroentanglement process, the material is dried. Preferably the moisture content is less than 20%, in particular less than 10%.

In a hydroentanglement process according to the present invention, the pressure of the water jets is preferably adjusted into the range of 80-250 bar for condensed structure, their form, jet number per area unit of the web, the forming process speed, and number of running cycles are selected such that the fibres of the blend are not cut during processing.

The fabric therefore exhibits properties of soft handling, drapability, smooth surface. Importantly, the fabric is essentially free, or shows only a small amount of staple fiber ends sticking out from the fabric surface. While the tensile properties go through a maximum at around 100 bar, the fabric extensibility shows a linear decrease with pressure. Continued consolidation of the fabric occurs in a pressure range of 100-250 bar as treatment proceeds above the breaking load optimum.

During the processing of the wound dressing by hydroentanglement the antimicrobial fibres can be evenly distributed throughout a fibrous matrix formed by the second fibres. Alternately, the antimicrobial fibres can be deposited on one side of such a fibrous matrix. FIG. 1 shows a wound dressing as discussed above, in which the first fibres are homogeneously distributed throughout a matrix formed by the second fibres, and FIG. 2 shows a wound dressing where the silver-coated fibres are located within one distinct layer.

The wound dressings can also be formed into multilayered structures.

In one embodiment of the invention, the fabric is a layered structure, wherein the first layer, capable of abutting the healing wound is comprised of a gauze or wire mesh formed by silver-coated fibres of the above-described kind and silver-free fibres capable of conferring properties of mechanical strength to the fabric. A second layer placed next to the first layer, on the opposite side to the healing wound, comprises a material capable of absorbing liquids, such as water. Such a layer can be formed by absorbing films or hydrogels or absorptive fibres.

The wound dressing according to the invention can be in the shape of an elongated dressing folded 1 to 10 times.

In the following calculation, the amount of silver and absorptive capacity reachable at various blend ratios of different fibres is given. These results can be used for producing, for example, a wound dress which comprises four overlapping layers of the present fabric and having properties of antimicrobial activity, liquid absorption and mechanical strength.

The first fibres are silver-coated polyamide fibres containing 30% of silver (wt) and the absorptive capacity (water retention) of the cotton/viscose fibres is 130%:

TABLE 1

| | Fibres | | | | |
|---|---|---|---|---|---|
| | AgPA:CV:PET | AgPA:CV:PET | AgPA:CV:PET | AgPA:CV:PET | AgPA:CV:PET |
| Blending ratio | 5:75:20 | 5:85:10 | 10:85:5 | 10:90:0 | 5:95:0 |
| (% by weight) | | | | | |
| Silver content, % | 1.5 | 1.5 | 3 | 3 | 1.5 |
| Water retention, % | 97.5 | 110 | 110 | 117 | 123.5 |

In the following the antimicrobial properties of the present materials are discussed based on test results. The test used is titled “Sterilization of medical devices—microbiological methods—Part 1: Determination of a population of micro-organisms on products”—EN ISO 11737-1:2006 (Bioburden).

### EXAMPLE

We performed the study as a function of time to detect the possible multiplication of microbes in textiles with silver.

The method employed (EN ISO 11737-1:2006) specifies the requirements and provides guidance for the enumeration and microbial characterization of the population of viable micro-organisms on or in a medical device, component, raw material or package. In short, the principle is the following:

Silver coated fibres of the kind mentioned above (30% elemental silver on PA fibres) were used.

Phase I

Under clean conditions (laminar flow cabinet or clean room) aseptically cut appropriate amount of samples (size 2 cm²) from the test material. According to the standard, as large portion of the material as possible should be tested (3 to 10 samples in routine bioburden level determinations).

The samples are placed in sterile Stomacher bags with appropriate amount of sterile culture liquid and run in a Stomacher apparatus for 3 minutes.

Using a membrane filtration method (0.45 u pore size filter), all samples are filtrated through the membrane. The filter is then aseptically transferred onto TSA-plates and all plates are incubated at 30 to 35° C. for 2 to 7 days. Colony counts are enumerated and recorded separately for each plate.

Phase 2

Samples like in previous test (phase 1) were contaminated with 1−5×10³ cfu/ml of methicillin-resistant Staphylococcus aureus (ATCC 43300). Then samples were dried at room temperature and incubated at +36° C. overnight (24 hrs). After that samples were handled as previously step 2-5. Test Report SFS-EN ISO 11737-1.

Test laboratory:Laboratory of microbiology and disinfection, Dept. of Public Health, University of Helsinki, Test period August 2010

Two different materials according to the present invention was use, viz. one which was composed of silver-coated polyamide fibres (AgPA) spunbonded with polyethylene terephthalate (PET) fibres and one with silver-coated polyamide fibres (AgPA) and cotton-viscose fibres (CV) spunbonded with polyethylene terephthalate (PET) fibres.

The results are indicated in Table 2:

TABLE 2

| | | Bioburden of samples | | | |
|---|---|---|---|---|---|
| Test | | Non- | Contaminated MRSA | | Bacterial |
| material, | Fiber | contaminated | 0 hrs, | 24 hrs, | reduction, |
| g/m² | content | count/sample | cfu/ml | cfu/ml | % |
| 1. Grey | AgPA 7%/ | 41/43/39 | 1.7 × 10 | 0/5/0 | >log 3 |
| textile | CV 80%/ | | exp4 | (<1 × 10 | |
| | PET 13% | | | exp1) | |
| 2. Black | AgPA 7%/ | 10/3/9 | 1.7 × 10 | 6/3/3 | >log 3 |
| textile | CV 0%/ | | exp4 | (<1 × 10 | |
| | PET 93% | | | exp1) | |
| 3. Textile | AgPA 7%/ | 74/65/50 | 1.7 × 10 | 1/2/1 | >log 3 |
| | CV 0%/ | | exp4 | (<1 × 10 | |
| | PET 93% | | | exp1) | |

Test method: stomaching and membrane filtration according to standard
Culture media: TSA (tryptic soy agar plates) and TSB (tryptic soy broth)
Test apparatus: Stomacher 80, Seward and sterile Stomacher bags

Conclusion: According to the standard, the present materials fulfill the standard requirements log cfu<2 up to 24 hours.

As known in the art, noble metals do not give rise to resistency among microbial strains. By regulating the amount of silver fibres in the bandage, it is possible to adjust the amount of silver ions (ppm) released for wound treatment.

1. An anti-microbial wound dressing comprising; a non-woven fabric produced by hydroentanglement of a blend of fibres, said blend being formed by first fibres which are coated with elemental silver or contain silver and second fibres which are essentially free from silver.

2. The wound dressing according to claim 1, wherein the first fibres comprise a core formed by a thermoplastic material, said core being coated with elemental silver or contain silver.

3. The wound dressing according to claim 1, wherein the first fibres comprise 0.1 to 75% by weight of elemental silver.

4. (canceled)

5. The wound dressing according to claim 1, wherein the second fibres are selected from the group of synthetic and natural, hydrophilic fibres, water absorbing films or hydrogels, hydrophobic fibres and combinations thereof.

6. (canceled)

7. The wound dressing according to claim 1, wherein the second fibres are reinforcing fibres selected from the group of polyolefins, polyacrylics, polyesters and polyamides and mixtures thereof and wherein the second fibres form 1 to 60% of the surface weight of the non-woven fabric.

8. (canceled)

9. The wound dressing according to claim 5, wherein the first fibres, optionally in combination with the second fibres, are formed into a gauze or wire mesh and wherein there is arranged in abutting relationship with the gauze or wire mesh a second layer comprising absorptive materials selected from absorbing films, hydrogel and absorptive fibres.

10. (canceled)

11. (canceled)

12. The wound dressing according to claim 1, wherein the linear density of the first fibres is 1 to 5 dtex and the linear density of the second fibres is 0.5 to 5 dtex.

13. (canceled)

14. (canceled)

15. The wound dressing according to claim 1, wherein the second fibres comprise absorptive fibres which form 10 to 99% of the surface weight of the non-woven fabric.

16. (canceled)

17. The wound dressing according to claim 1, wherein the first fibres have an average fibre length of 3 to 100 mm and are essentially free from staple fibres.

18. (canceled)

19. The wound dressing according to claim 1, wherein the surface weight of the non-woven fabric is 30 to 200 g/m².

20. (canceled)

21. The wound dressing according to claim 1, wherein the first fibres are homogeneously distributed throughout a matrix formed by the second fibres.

22. The wound dressing according to claim 1, wherein elemental silver is capable of being liberated from the fibre surface of the first fibres to the surrounding at a release rate of 1 to 100 ppm/day.

23. An anti-microbial wound dressing comprising; a non-woven fabric produced by hydroentanglement of a blend of fibres, said blend being formed by first fibres which are coated with elemental copper or contain copper and second fibres which are essentially free from copper.

24. The wound dressing according to claim 1, wherein the first fibres comprise a combination of silver and copper.

25. A method of producing an anti-microbial wound dressing comprising a non-woven fabric, comprising the steps of
providing first fibres coated with elemental silver or contain silver;
providing second fibres which are essentially free from silver;
forming a blend of said first and second fibres; and
producing a non-woven fabric from said blend by hydroentanglement.

26. The method according to claim 25, wherein the step of providing first fibres comprising depositing on thermoplastic fibres a coating of elemental silver.

27. The method according to claim 26, wherein elemental silver is deposited on thermoplastic fibres by electrolytic or chemical precipitation from a solution or where silver is introduced by using master-batch manufacturing and fiber spinning methods.

28. (canceled)

29. The method according to claim 25, wherein the blend of first and second fibres is subjected to hydroentanglement to produce a non-woven fabric in which the second fibres form the matrix, in which the first fibres are homogeneously distributed.

30. The method according to claim 25, comprising providing
1 to 60 parts by weight of first fibres;
forming a blend of said first and said second fibres into a fibrous web; and
subjecting said fibrous web to hydroentanglement.

31. (canceled)

32. (canceled)
